(19) United States
(12) Patent Application Publication
(10) Pub. No.: US 2025/0272998 A1
(43) Pub. Date: Aug. 28, 2025

(54) BONY FEATURE DETECTION USING IMAGE SEGMENTATION

(71) Applicant: University of Florida Research Foundation, Incorporated, Gainesville, FL (US)

(72) Inventors: Sanjeev KUMAR, Gainesville, FL (US); Taylor Kristian CALIBO, Gainesville, FL (US)

(21) Appl. No.: 19/057,615

(22) Filed: Feb. 19, 2025

Related U.S. Application Data
(60) Provisional application No. 63/558,156, filed on Feb. 27, 2024.

(51) Int. Cl. G06V 20/70; A61B 34/20; G06V 10/26; G06V 10/774

(52) U.S. Cl. CPC G06V 20/70; A61B 34/20; G06V 10/26; G06V 10/774; G06V 2201/033; G06V 2201/034

(57) ABSTRACT

A medical visualization is provided by providing a medical image in a particular image format to an input of a trained object detection model; causing the model to be executed to process the medical image to generate processed image information, the processed image information identifies bony features within the medical image; receiving the processed image information from an output of the model; and causing display of the medical image with a representation of the processed image information overlaid on the medical image. The model was trained using a training process comprising performing instance segmentation on a plurality of training images, each of the training images in the particular image format; assigning semantic labels to objects identified via the instance segmentation; and using the training images, the instance segmentation of the training images, and the semantic labels assigned to objects identified in the training images to train an object detection model.

### CROSS-REFERENCE TO RELATED APPLICATION(S)

This application claims priority to U.S. Application No. 63/558,156, filed Feb. 27, 2024, the content of which is incorporated herein by reference in its entirety.

### FIELD

Various embodiments relate to detecting bony features and, possibly, medical instruments in medical images. An example embodiment relates generally to the detection of bony features and, possibly, medical instruments in medical images and generating predictions based on the detected features.

### BACKGROUND

Lumbar transforaminal epidural steroid injections, guided by fluoroscopy, are a mainstay in managing lumbar pain among interventional anesthesiologists in the United States. They are lauded for their effectiveness in short-term pain relief. However, patients with lumbar spine degeneration and stenosis often present complexities that demand greater precision in the procedure. The potential for complications, although generally minor, such as vasovagal reactions, pain exacerbation, and injection site soreness, underscores a critical need for enhanced accuracy in needle placement. This accuracy is not just a matter of improving the procedure's effectiveness but is also a consideration for patient safety. Through applied effort, ingenuity, and innovation many deficiencies of prior guidance techniques have been solved by developing solutions that are structured in accordance with the embodiments of the present invention, many examples of which are described in detail herein.

### BRIEF SUMMARY OF VARIOUS EMBODIMENTS

Various embodiments provide methods, apparatuses, systems, computer program products, and/or the like for detecting bony features in medical images. In various embodiments, the medical images are fluoroscopy images, ultrasound images, magnetic resonance imaging (MRI) images, and/or other medical images. In various embodiments, a medical device, such as an epidural needle, is also detected within the medical image. Various predictions regarding a trajectory previously traversed by a medical device, a future trajectory of a medical device, a goal trajectory of a medical device, a most advantageous level for medical intervention, and/or the like may be determined based on the medical image. In some embodiments, the medical image is one of a time series of medical images. In some embodiments, the medical image is an oblique contralateral image.

In various embodiments, a computing entity provides a medical image in a particular image format to an input of a trained object detection model and causes the trained object detection model to be executed to process the medical image to generate processed image information. The processed image information is configured to at least identify one or more bony features represented within the medical image. The computing entity receives the processed image information from an output of the trained object detection model; and causes display of the medical image with a representation of the processed image information overlaid on the medical image. The trained object detection model was trained using a training process comprising performing instance segmentation on a plurality of training images, each of the plurality of training images being in the particular image format; assigning semantic labels to teach object identified via the instance segmentation; and using at least a portion of the plurality of training images on which the instance segmentation has been performed, the corresponding objects, and the semantic labels to train an object detection model.

According to an aspect, a method for providing a medical visualization is provided. The method includes providing, by one or more processors of a computing entity, a medical image in a particular image format to an input of a trained object detection model; causing, by the one or more processors, the trained object detection model to be executed to process the medical image to generate processed image information, the processed image information configured to at least identify one or more bony features within the medical image; receiving, by the one or more processors, the processed image information from an output of the trained object detection model; and causing, by the one or more processors, display of the medical image with a representation of at least a portion of the processed image information overlaid on the medical image. The trained object detection model was trained using a training process comprising performing instance segmentation on a plurality of training images, each of the plurality of training images being in the particular image format; assigning semantic labels to each object identified via the instance segmentation; and using at least a portion of the plurality of training images, the instance segmentation of the at least a portion of the plurality of training images, and the semantic labels assigned to objects identified in the at least a portion of the plurality of training images to train an object detection model.

According to another aspect, a computing entity configured to provide a medical visualization is provided. The computing entity comprises at least one processor and a memory. The memory stores computer executable instructions configured to, when executed by the at least one processor, cause the computing entity to perform providing a medical image in a particular image format to an input of a trained object detection model; causing the trained object detection model to be executed to process the medical image to generate processed image information, the processed image information configured to at least identify one or more bony features within the medical image; receiving the processed image information from an output of the trained object detection model; and causing display of the medical image with a representation of at least a portion of the processed image information overlaid on the medical image. The trained object detection model was trained using a training process comprising performing instance segmentation on a plurality of training images, each of the plurality of training images being in the particular image format; assigning semantic labels to each object identified via the instance segmentation; and using at least a portion of the plurality of training images, the instance segmentation of the at least a portion of the plurality of training images, and the semantic labels assigned to objects identified in the at least a portion of the plurality of training images to train an object detection model.

According to still another aspect, a computer program product configured to cause a computing entity to provide a medical visualization is provided. The computer program product comprises at least one non-transitory computer-readable medium. The computer-readable medium stores computer-executable instructions configured to, when executed by a processor of a computing entity, cause the computing entity to perform providing a medical image in a particular image format to an input of a trained object detection model; causing the trained object detection model to be executed to process the medical image to generate processed image information, the processed image information configured to at least identify one or more bony features within the medical image; receiving the processed image information from an output of the trained object detection model; and causing display of the medical image with a representation of at least a portion of the processed image information overlaid on the medical image. The trained object detection model was trained using a training process comprising performing instance segmentation on a plurality of training images, each of the plurality of training images being in the particular image format; assigning semantic labels to each object identified via the instance segmentation; and using at least a portion of the plurality of training images, the instance segmentation of the at least a portion of the plurality of training images, and the semantic labels assigned to objects identified in the at least a portion of the plurality of training images to train an object detection model.

### BRIEF DESCRIPTION OF THE DRAWINGS

Having thus described certain example embodiments in general terms, reference will hereinafter be made to the accompanying drawings, which are not necessarily drawn to scale, and wherein:

FIG. 1 is a block diagram showing an example architecture of an example embodiment of a medical image-based information system;

FIG. 2A is a data flow diagram illustrating various features of the training of an object detection model, in accordance with an example embodiment;

FIG. 2B is a data flow diagram illustrating various features of using a trained object detection model to generate and provide processed image information, in accordance with an example embodiment;

FIG. 3 is a flowchart illustrating various processes and/or procedures performed by a computing entity of FIG. 6, for example, for training an object detection model, in accordance with an example embodiment;

FIGS. 4A and 4B illustrate an example medical image and segments identified and/or labeled therein, in accordance with an example embodiment;

FIG. 4C illustrates an example medical image with a graphical representation of at least a portion of the processed image information overlaid thereon, in accordance with an example embodiment; and

FIG. 5 is a flowchart illustrating various processes and/or procedures performed by a computing entity of FIG. 6, for example, for providing processed image information, in accordance with an example embodiment.

### DETAILED DESCRIPTION OF SOME EXAMPLE EMBODIMENTS

### I. General Overview

Various embodiments provide methods, apparatuses, systems, computer program products, and/or the like for extracting information from medical images. In various embodiments, the information extracted from a medical image includes locations of bony features and, possibly medical devices, in the medical image. In various embodiments, the medical images are fluoroscopy images, ultrasound images, magnetic resonance imaging (MRI) images, and/or other medical images. In an example embodiment, information is extracted from a time series of medical images (e.g., a series of medical images taken over a period of time and organized in a time-ordered sequence).

In various embodiments, a medical device, such as an epidural needle, is also detected within the medical image. Various predictions regarding a trajectory previously traversed by a medical device, a future trajectory of a medical device, a goal trajectory of a medical device, a most advantageous level for medical intervention, and/or the like may be determined based on the medical image. In some embodiments, the medical image is an oblique contralateral image.

In various embodiments, a computing entity provides a medical image in a particular image format to an input of a trained object detection model. In an example embodiment, the particular image format is a raster-graphics file format that supports lossless data compression, such as .png. The computing entity causes the trained object detection model to be executed to process the medical image to generate processed image information. The processed image information is configured to at least identify one or more bony features represented within the medical image. In some instances, the processed image information is further configured to identify a medical device (e.g., an epidural needle) resent in the medical image; a past, future, and/or goal trajectory for the medical device; a most advantageous level for medical intervention; and/or the like. The computing entity receives the processed image information from an output of the trained object detection model; and causes display of the medical image with a representation of the processed image information overlaid on the medical image. In various embodiments, the representation of the processed image information overlaid thereon is displayed in real-time with respect to the capturing of the medical data (e.g., during a procedure that includes the insertion of the medical device into a patient).

In various embodiments, the trained object detection model is trained using a training process comprising obtaining a plurality of training images in the particular image format and performing instance segmentation on a plurality of training images. In various embodiments, training the object detection model further includes assigning semantic labels to teach object identified via the instance segmentation. For example, the semantics labels may indicate whether the labeled object is a medical device, a bony feature, and/or the like. In various embodiments, training the object detection model further includes using at least a portion of the plurality of training images on which the instance segmentation has been performed, the corresponding objects, and the semantic labels to train the object detection model. For example, various machine learning algorithms may be used to train the object detection model based at least in part on the plurality of training images on which the instance segmentation has been performed, the corresponding objects, and the semantic labels. The trained object detection model may then be used to process medical images to extract information therefrom, generate predictions based thereon, and/or the like.

### II. Example System Architecture

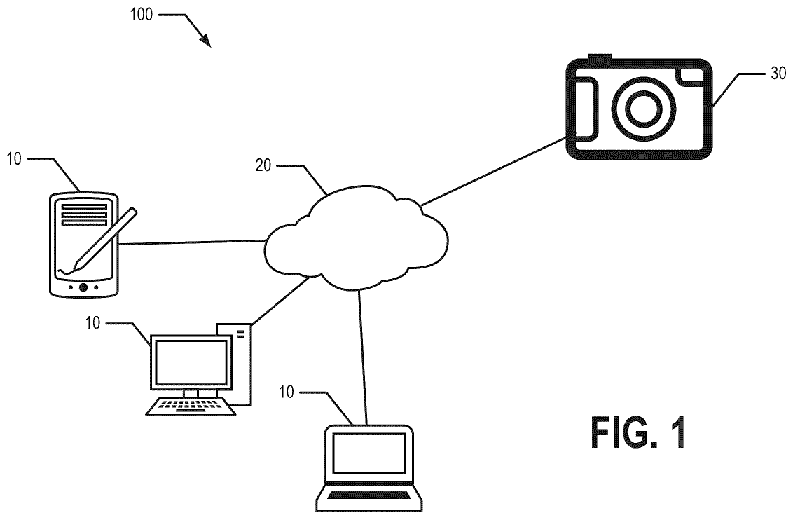

FIG. 1 provides an illustration of an example block diagram of an example medical image-based information system 100 that can be used in conjunction with various embodiments of the present invention. As shown in FIG. 1, the medical image-based information system 100 may include one or more computing entities 10 and may include one or more image capture devices 30. In various embodiments, the one or more image capture devices 30 are in communication with the one or more computing entities 10 via one or more wired and/or wireless networks 20.

In various embodiments, the computing entity 10 is a server, server bank, part of a cloud-based computing environment, desktop computer, laptop, tablet, smartphone, other computing device and/or the like. In various embodiments the computing entity 10 is configured to train and/or execute an object detection model. For example, the computing entity 10 may be configured to train an object detection model via one or more machine learning algorithms such that the object detection model is configured to detect bony features and, possibly, medical devices within a medical image. In various embodiments, the computing entity 10 is configured to train the object detection model to predict a past, future, and/or goal trajectory of a medical device; a most advantageous level for medical intervention; and/or the like. In various embodiments, the computing entity 10 is configured to execute the object detection model to process a medical image and/or time series of medical images to detect one or more bony features and, possibly, medical devices in the medical image(s) and/or to determine one or more predictions.

In various embodiments, the image capture device 30 is an X-ray imaging device, ultrasound imaging device, MRI device, and/or other device configured for capturing medical images. In various embodiments, the image capture device 30 is configured to capture and/or generate one or more medical images. In an example embodiment, the image capture device 30 is configured to capture and/or generate one or more medical images of patient during the performance of a medical procedure. For example, in an example embodiment, the image capture device 30 is configured to capture fluoroscopic images, including oblique contralateral images, of a patient while an epidural is being administered to the patient.

In various embodiments, the image capture device 30 generates and/or captures one or more medical images and provides the medical image(s) such that the computing entity 10 receives the medical image(s). For example, the image capture device 30 may be in wired and/or wireless communication with a computing entity 10.

For example, in an example embodiment, a computing entity 10 may comprise components similar to those shown in the example computing entity 10 diagrammed in FIG. 6. In an example embodiment, the computing entity 10 is configured to train the object detection model. In an example embodiment, the computing entity 10 is configured to receive obtain training images, perform segmentation of the training images, label the segmented training images, and train the object detection model based thereon using a machine learning algorithm, and/or the like. In an example embodiment, the computing entity 10 is configured to obtain medical images and cause the trained object detection model to process the medical images to detect bony features and, possibly, medical devices within the medical images, generate predictions based on the medical images, and/or the like. In various embodiments, the computing entity 10 is configured to cause display of a medical image with a graphical representation of processed image information overlaid thereon.

For example, as shown in FIG. 6, the computing entity 10 may comprise a processor 102, memory 104, a user interface 108, a communication interface 106, and/or other components configured to perform various operations, procedures, functions or the like described herein. In at least some example embodiments, the memory 104 is non-transitory. Certain example embodiments of the computing entity 10 are described in more detail below with respect to FIG. 6.

### III. Exemplary Operation of a Medical Image-Based Information System

Various embodiments provide methods, apparatuses, systems, computer program products, and/or the like for extracting information from medical images. In various embodiments, the information is extracted from the medical images using an object detection model. In various embodiments, the object detection model includes a feature detector configured to identify various features within medical images (e.g., detection of bony features and/or medical devices in medical images). In some embodiments, the object detection model further includes a prediction model configured to generating predictions based on the features identified in one or more medical images. The object detection model is trained using a machine learning algorithm.

### A. Example Training of an Object Detection Model

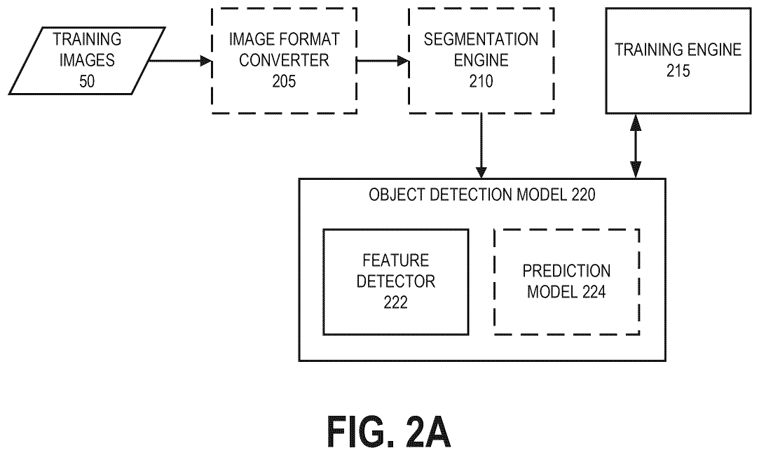

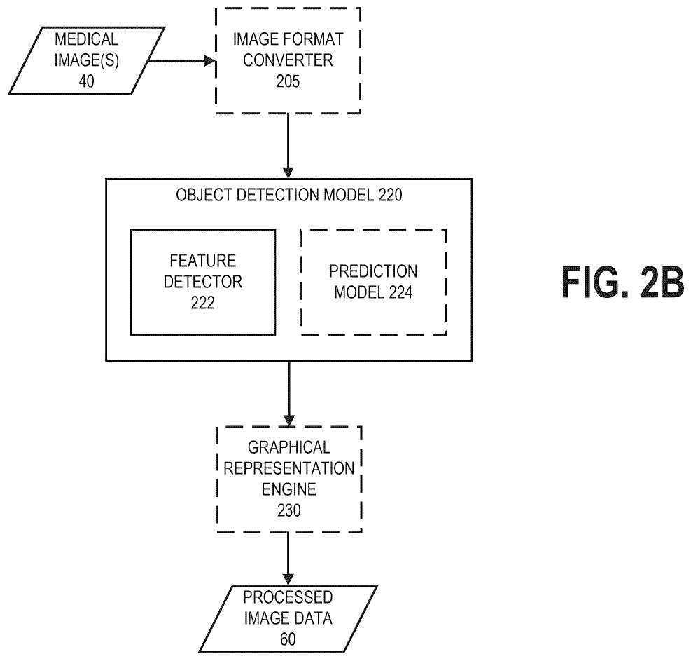

FIG. 2A provides a data flow diagram of operations performed by a computing entity 10 to train an object detection model 220. For example, a computing entity 10 obtains a plurality of training images 50. The computing entity 10 may comprise an image format converter 205. For example, the computing entity 10 may store, in a memory thereof, executable instructions and/or program code configured to, when executed by a processor of the computing entity 10, provides an image format converter 205. In an example embodiment, the image format converter 205 is configured to convert training images from an initial image format to the particular image format. In an example embodiment, the plurality of training images 50 are obtained being already in the particular image format, and the computing entity 10 may not include or may not use the image format converter 205 in the training of the object detection model 220.

The plurality of training images 50 in the particular image format are provided to a segmentation engine 210. For example, the computing entity 10 may store, in a memory thereof, executable instructions and/or program code configured to, when executed by a processor of the computing entity 10, provides a segmentation engine 210. In an example embodiment, the segmentation engine 210 is configured to perform instance segmentation on the plurality of training images 50 automatically (e.g., without requiring user input for direct use in segmenting at least one of the training images). For example, in an example embodiment, the segmentation engine 210 comprises a trained neural network, such as a generative adversarial network, and/or the like configured to perform instance segmentation of medical images. In an example embodiment, the segmentation engine 210 is configured to provide a graphical user interface (GUI) to assist a user in providing user input to perform segmentation of at least one of the training images. In various embodiments, the segmentation engine 210 is configured to assign (automatically or via user input) semantic labels to each segment or object within a training image. For example, an object identified in a training image may be labeled so as to indicate whether the object is a bony feature, what type of bony feature the object is (e.g., a pedicle of a vertebra, an end plate of a vertebra, etc.), whether the object is a medical device, and/or the like. The segmented and labeled training images are then provided to the object detection model 220. In various embodiments, the segmentation engine 210 comprises a computer vision model trained to perform segmentation and/or labeling of medical images.

In some embodiments, the training images may be labeled with a trajectory. For example, a set of training images of the plurality of training images may be a time series of medical image including a medical device (e.g., an epidural needle, and/or the like) with at least one of the medical images of the time series of medical images labeled with the trajectory of the medical device and/or labeled with an indication of whether the trajectory is a good trajectory (e.g., reaches its goal location, is considered safe, and/or the like) or not a good trajectory (e.g., does not reach its goal location, is not considered safe, and/or the like).

In various embodiments, the computing entity 10 comprises a training engine 215. For example, the computing entity 10 may store, in a memory thereof, executable instructions and/or program code configured to, when executed by a processor of the computing entity 10, provides a training engine 215. In various embodiments, the training engine 215 is configured to train an object detection model 220 using the plurality of training images, the segmentation thereof, and labels associated and/or assigned thereto. In various embodiments, the training engine 215 may be configured to perform various machine learning algorithms, such as gradient descent, and/or the like. For example, the object detection model 220 may include a convolution neural network and/or other neural network and the training engine 215 is configured to train the convolution neural network and/or other neural network of the object detection model 220.

For example, the training engine 215 provides training images to the object detection model 220 such that the training images (e.g., either as single medical images or time series of medical images) are processed via the training engine 215 such that the object detection model 220 learns to identify bony features and any medical devices present in a medical image via the feature detector 222. In various embodiments, the object detection model 220 includes a prediction model 224 in addition to the feature detector 222. In various embodiments, the feature detector 222 and/or the prediction model 224 include respective neural networks and/or artificial intelligence (AI) models. For example, in an example embodiment, the feature detector 222 comprises a convolution neural network (CNN) and/or the like. In some embodiments, the prediction model 224 comprises a CNN, generative adversarial network (GAN), various types of classifiers, and/or the like.

In various embodiments, the training engine 215 is configured to train the prediction model 224 to generate one or more predictions regarding the trajectory of a medical device (e.g., a past trajectory, future trajectory, a goal trajectory that is a good trajectory, whether the past and/or future trajectory is good trajectory or not, and/or the like). In various embodiments, the training engine 215 is configured to train the prediction model 224 to identify a most advantageous level for medical intervention, a goal location for a trajectory of the medical device, and/or the like. In various embodiments, the prediction model 224 is configured to receive an output of the feature detector 222 as input.

The feature detector 222, the prediction model 224, and/or other portions of the object detection model 220 may be iteratively trained via the training engine 215 such that the weights, parameters, and/or the like of the feature detector 222, the prediction model 224, and/or other portions of the object detection model 220 are determined, learned, and/or the like. In various embodiments, the feature detector 222 and/or the prediction model 224 include respective neural networks and/or artificial intelligence (AI) models.

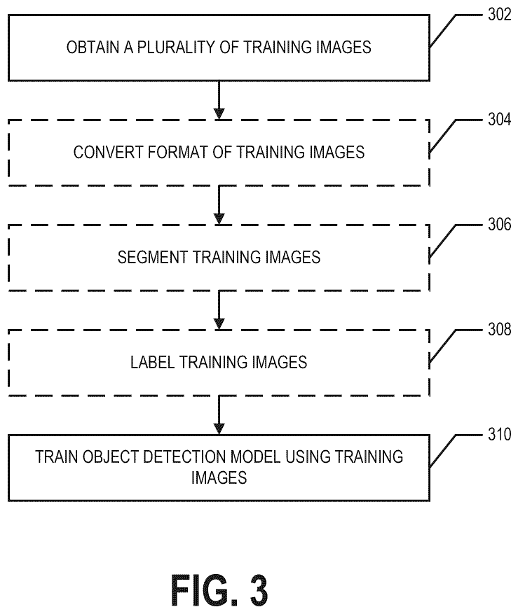

FIG. 3 provides a flowchart illustrating various processes, procedures, and/or operations performed by a computing entity 10, an example embodiment of which is illustrated in FIG. 6, for training an object detection model 220.

Starting at step 302, a plurality of training images 50 are obtained. For example, a plurality of training images is stored in a database. The database may be stored by the computing entity 10 (e.g., in memory 104) or by another computing entity in communication with the computing entity 10 (e.g., via network 20). For example, the computing entity 10 comprises means, such as processor 102, memory 104, communication interface 106, and/or the like, for obtaining a plurality of training images. For example, the plurality of training images may be read and/or accessed from memory, received via the communication interface 106, and/or the like. In various embodiments, the plurality of training images comprises a plurality of medical images.

In various embodiments, the plurality of training images comprises one or more time series of medical images. In various embodiments, each of the medical images of the plurality of training images are of a same type of medical images (e.g., all fluoroscopy images, all ultrasound images, all MRI images, and/or all another type of medical images). In various embodiments, the plurality of training images may include two or more types of medical images. In various embodiments, the plurality of training images includes medical images all of the same perspective (e.g., oblique contralateral, lateral, posterior, anterior, and/or the like). In various embodiments, the plurality of training images includes medical images of two or more perspectives.

At step 304, the computing entity 10 converts the plurality of training images 50 to the particular image format. In various embodiments, the medical images of the plurality of training images are obtained in an initial format. The computing entity 10 may determine and/or confirm whether the initial format of the plurality of training images 50 is the particular image format. For example, the computing entity 10 (e.g., the image format converter 205) may compare a format name, file extension, metadata, the formatting of the image data, and/or the like of one or more of the medical images of the plurality of training images to a corresponding property of the particular image format. When the corresponding properties of the plurality of training images and the particular image format match, the computing entity 10 determines that the initial format is the particular image format, and no conversion is necessary. When the corresponding properties of the plurality of the training images and the particular image format do not match, the computing entity 10 determines that the initial format is not the particular image format and may cause conversion of the plurality of training images to the particular image format. For example, when the initial format of the plurality of training images is not the particular image format, the computing entity 10 may execute the image format converter 205 to cause the plurality of training images 50 to be converted to the particular image format. For example, the computing entity 10 comprises means, such as processor 102, memory 104, and/or the like, for converting the plurality of training images from an initial format to the particular image format.

In an example embodiment, the particular image format is a raster-graphics file format that supports lossless data compression, such as .png.

At step 306, the computing entity 10 segments the plurality of training images 50. For example, in various embodiments, the computing entity 10 comprises means, such as the processor 102, memory 104, user interface 108, and/or the like for segmenting the plurality of training images 50. For example, in an example embodiment, the computing entity 10 executes the segmentation engine 210 to cause the segmentation engine 210 to segment the plurality of training images 50. In an example embodiment, the segmentation engine 210 comprises a machine learning trained segmentation model configured to perform automated segmentation of the plurality of training images. In an example embodiment, the segmentation engine 210 is configured to provide a GUI (e.g., via the user interface 108) configured to receive user input identifying one or more segments within a medical image of the plurality of training images. In various embodiments, the segmentation engine 210 is configured to perform instance segmentation. For example, the plurality of training images may be segmented using an instance segmentation algorithm or technique.

At step 308, the segments of the plurality of training images are labelled. For example, the computing entity 10 may assign a semantic label to each segment of the plurality of training images. For example, the computing entity 10 comprises means, such as processor 102, memory 104, user interface 108, and/or the like, for assigning a semantic label to each segment of the plurality of training images. As used herein a semantic label is configured to provide an indication of what a segment or object is or represents, rather than merely identifying the segment or object within a plurality of segments or objects. For example, in various embodiments, the segmentation engine 210 is configured to assign (automatically or via user input) semantic labels to each segment within a medical image of the plurality of training images. For example, a segment identified in a medical image of the plurality of training images 50 may be labeled so as to indicate whether the object is a bony feature, what type of bony feature the object is (e.g., a pedicle of a vertebra, an end plate of a vertebra, etc.), whether the object is a medical device, a type of the medical device, and/or the like.

In some embodiments, the training images may be labeled with a trajectory. For example, a set of training images of the plurality of training images may be a time series of medical images including a medical device (e.g., an epidural needle, and/or the like) with at least one of the medical images of the time series of medical images labeled with the trajectory of the medical device and/or labeled with an indication of whether the trajectory is a good trajectory (e.g., reaches its goal location, is considered safe, and/or the like) or not a good trajectory (e.g., does not reach its goal location, is not considered safe, and/or the like).

In various embodiments, steps 306 and 308 are performed at the same time. For example, a medical image of the plurality of training images 50 may be simultaneously segmented and labeled. For example, the segmentation and labeling may be performed image by image. In some embodiments, steps 306 and 308 are performed in series. For example, each of the medical images of the plurality of training images 50 may be segmented and then each of the medical images of the plurality of training images 50 may be labeled. In some embodiments, the plurality of training images is already segmented and labeled when they are obtained (e.g., steps 306 and 308 may be performed by the computing entity 10 or by another computing entity prior to step 302).

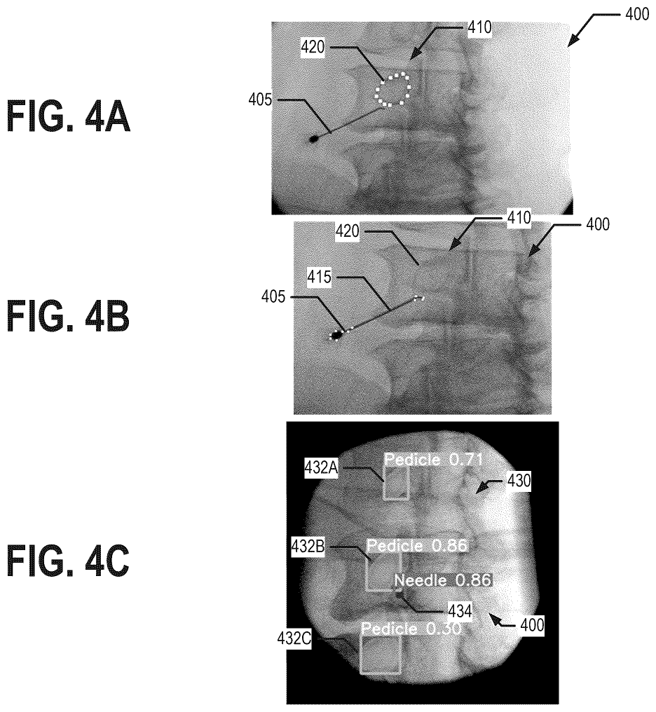

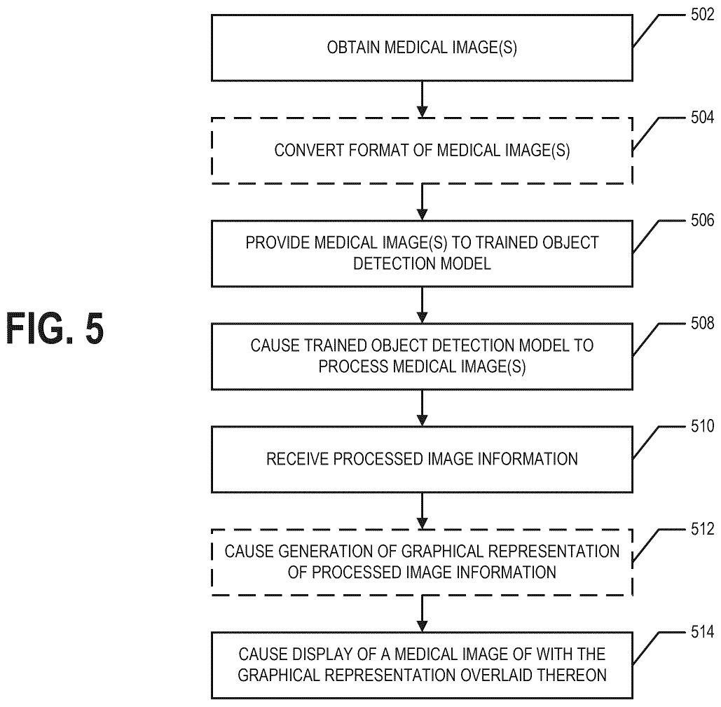

FIGS. 4A and 4B illustrate an example medical image 400 of the plurality of training images 50. The example medical image 400 is an oblique fluoroscopic image including at least one vertebral pedicle 410 and a medical device 405. FIG. 4A includes a polygon 420 illustrating the border defining a segmentation of the vertebral pedicle 410 within the medical image 400. In various embodiments, the bony features (e.g., vertebral pedicle, vertebral end plates, and/or the like) are enclosed by irregular polygons.

FIG. 4B includes a polygon 415 illustrating the border defining a segmentation of the medical device 405 within the medical image 400. For example, in an example embodiment, objects within a medical image of the plurality of images are segmented by defining and/or selecting one or more points that define a (smallest) polygon enclosing the object. In various embodiments, the medical device is enclosed by a polygon having a large length to width ratio. For example, one dimension of the (smallest) polygon enclosing a medical device is defined along a principal axis thereof and/or along a longest dimension thereof. A second dimension of the (smallest) polygon enclose the medical device is defined along a different axis of the polygon that is perpendicular to the principal axis or longest dimension. A length of the polygon in the first dimension is larger than a width of the polygon in the second dimension by a factor of two, three, five, ten, or more. For example, the ratio of the length of the polygon in the first dimension to the width of the polygon in the second dimension is at least two, three, five, or ten, in various embodiments.

The polygons 415, 420 are associated with and/or assigned semantic labels. The polygons and associated/assigned semantic labels are stored in association with the corresponding medical image of the plurality of training images. The segmented and labeled plurality of training images are then provided to the object detection model 220 (e.g., via the training engine 215, in an example embodiment) such that the object detection model 220 is trained using at least a portion of the plurality of training images and corresponding segmentation and labels.

At step 310, the training engine 215 trains the object detection model 220 using at least some of the plurality of training images and the corresponding segmentations and labels. For example, the computing entity 10 comprises means, such as processor 102, memory 104, and/or the like, for training the object detection model 220 using at least some of the plurality of training images and the corresponding segmentations and labels. For example, the training engine 215 may be configured to provide a first portion of the plurality of training images and corresponding segmentations and labels to the object detection model 220 such that the object detection model may learn to identify objects within medical images, classify the objects identified within the medical images, and, possibly, generate one or more predictions based thereon. The training engine 215 may then provide a second portion of the plurality of training images to the object detection model 220 (without the segmentations and labels corresponding thereto) and compare the output of the object detection model 220 to the segmentations and labels corresponding to the second portion of the plurality of training images to perform a validation of the training of the object detection model 220. The object detection model 220 may be iteratively trained (e.g., via a gradient descent algorithm, and/or the like), may be pre-trained and then trained, and/or may be re-trained using at least some of the plurality of training images 50.

In various embodiments, the object detection model 220 comprises a feature detector 222 that is trained to identify objects within a medical image. For example, the feature detector 222 may be trained to spatially identify objects within a medical image and to determine semantic labels (e.g., a bony feature, medical device, and/or the like) for the identified objects. For example, the feature detector 222 may identify and classify objects within the medical images. In various embodiments, the feature detector 222 is configured to identify and classify multiple classes of objects within a single medical image. In an example embodiment, the feature detector 222 uses a two-step algorithm such as a first step to identify objects within a medical image and a second step to classify the identified objects. In an example embodiment, the feature detector 222 uses a single-step algorithm such as a single step to both identify and classify objects within a medical image. For example, in an example embodiment, the feature detector 222 may be a version of You Only Look Once (YOLO) and/or a similar computer vision model (e.g., convolution neural network and/or the like).

For example, the training engine 215 provides a first portion of the plurality of training images and the associated segmentations and labels to the object detection model 220 such that the training images (e.g., either as single medical images or time series of medical images) are processed via the training engine 215 such that the object detection model 220 learns to identify and classify bony features and any medical devices present in a medical image via the feature detector 222.

In various embodiments, the object detection model 220 further includes a prediction model 224. In various embodiments, the training engine 215 is configured to train the prediction model 224 to generate one or more predictions regarding the trajectory of a medical device (e.g., a past trajectory, future trajectory, a goal trajectory that is a good trajectory, whether the past and/or future trajectory is good trajectory or not, and/or the like). In various embodiments, the training engine 215 is configured to train the prediction model 224 to identify a most advantageous level for medical intervention, a goal location for a trajectory of the medical device, and/or the like. In various embodiments, the prediction model 224 is configured to receive an output of the feature detector 222 as input.

For example, in an example embodiment, the feature detector 222 is trained to identify vertebral end plates in a medical image (e.g., an oblique fluoroscopy image). The polygons generated by the feature detector 222 that locate the vertebral end plates in the medical image (possibly along with corresponds labels/classifications) and the prediction model 224 may be trained to identify a most degenerated vertebra or vertebral end plate based on distances between vertebral end plates, a measure or degree of irregularity of the vertebral end plates, and/or the like. For example, the vertebral end plate having the greatest measure or degree of irregularity of the vertebral end plates in the medical image may be determined to be most advantageous level for medical intervention, a goal location for a trajectory of a medical device (e.g., an epidural needle), and/or the like.

In another example, the prediction model 224 is trained to determine a past trajectory of a medical device (e.g., a trajectory the medical device travelled to reach its current position), a future trajectory of the medical device (e.g., the trajectory the medical device will travel unless a change is made in how the medical device is being pushed), a goal trajectory of the medical device (e.g., a trajectory the medical device should follow to reach the goal location), and/or the like. In various embodiments, the prediction model 224 is configured to classify a trajectory as to whether the trajectory is a good trajectory (e.g., will reach the goal location, is considered safe for the patient, and/or the like) or not a good trajectory (e.g., likely will not reach the goal location, is not considered safe for the patient, and/or the like).

Once the object detection model 220 is trained, the object detection model may be used to process medical images and, possibly, provide real-time feedback and/or visualizations regarding medical features actively being performed. In some embodiments, the object detection model 220 is used to provide feedback and/or visualizations prior to the beginning of a medical procedure, after completion of a medical procedure, to determine whether a medical procedure is likely to be helpful to a patient, and/or the like.

### B. Example Use of an Object Detection Model

Once the object detection model 220 has been trained, the object detection model may be used to extract information from medical images. For example, a computing entity 10 (e.g., the same computing entity 10 used to train the object detection model or a different computing entity 10) may execute the trained object detection model to cause the object detection model to extract information from one or more medical images.

FIG. 2B provides a data flow diagram of operations performed by a computing entity 10 to use an object detection model 220 to provide a medical visualization. One or more medical images 40 corresponding to a patient are obtained. For example, the one or more medical images 40 may be a medical image 40 corresponding to a patient, a set of medical images corresponding to the patient, and/or a time series of medical images corresponding to the patient are obtained. In various embodiments, the one or more medical images 40 are fluoroscopy images, ultrasound images, magnetic resonance imaging (MRI) images, and/or other medical images.

When the one or more medical images 40 are not in the particular image format when they are obtained, the computing entity 10 converts the one or more medical images 40 to the particular image format using the image format converter 205.

The one or more medical images 40 in the particular image format are provided as input to the object detection model 220. The computing entity 10 causes the object detection model 220 to process the one or more medical images 40 to extract processed image information therefrom. For example, the object detection model 220 is configured to transform the image data of the one or more medical images 40 (in the particular image format) into processed image information 60. For example, the processed image information may provide localization information and classification information for one or more objects identified and/or detected within the one or more medical images 40. For example, the processed image information may include a prediction regarding the medical images 40 generated by the prediction model 224 based on the provide localization information and classification information for one or more objects identified and/or detected within the one or more medical images 40. For example, the processed image information may include a past, future, and/or goal trajectory, an indication of whether the trajectory is a good or not good trajectory, a goal location for a medical device to reach, a most advantageous for medical intervention, and/or the like.

In various embodiments, the computing entity 10 comprises a graphical representation engine 230. For example, the computing entity 10 may store, in a memory thereof, executable instructions and/or program code configured to, when executed by a processor of the computing entity 10, provides a graphical representation engine 230. In various embodiments, the graphical representation engine 230 is configured to generate a graphical representation of the processed image information. For example, the graphical representation engine 230 may generate and/or render a graphical representation of the processed image information. In various embodiments, the rendered graphical representation of the processed image information is an image layer configured to be overlaid on at least one of the one or more medical images 40.

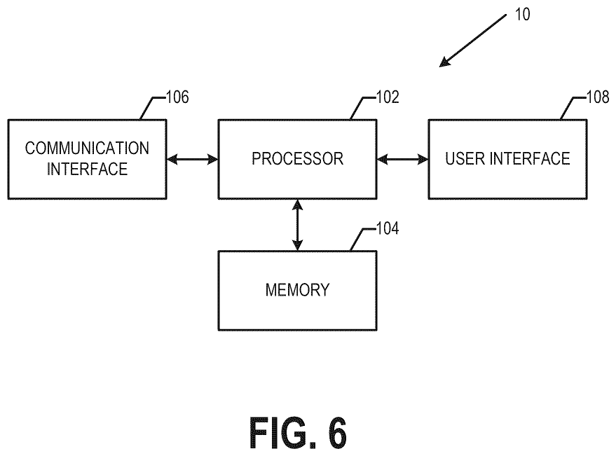

FIG. 5 provides a flowchart of various processes, procedures, and/or operations performed by a computing entity 10, for example, to provide a medical visualization corresponding to one or more medical images. The computing entity 10 that performs the steps illustrated in FIG. 5 may be the same computing entity or a different computing entity than the computing entity that performs the steps illustrated in FIG. 3. For example, in an example embodiment, a computing entity 10 trains the object detection model 220 and the same computing entity 10 is then used to provide medical visualizations using the object detection model 220. In another example embodiment, a computing entity such as server, desktop, laptop, and/or the like trains the object detection model 220. The object detection model 220 is then packaged into an application and distributed. For example, another computing entity 10, such as a smartphone, laptop, tablet, in-operating-room visualization and/or data system, and/or the like, may operate the application including the object detection model 220 to provide the medical visualizations. For example, the application may be configured to provide real-time medical visualizations during performance of a medical procedure.

Starting at step 502, computing entity 10 obtains one or more medical images. In an example embodiment, the one or more medical images are obtained by accessing and/or reading the medical images 40 from memory 104. In an example embodiment, the one or more medical images 40 are obtained by receiving the one or more medical images 40. For example, an image capture device 30 may capture one or more medical images and provide the medical images such that the computing entity 10 receives the medical images (e.g., via network 20). For example, the computing entity 10 comprises means, such as processor 102, memory 104, communication interface 106, and/or the like, for obtaining one or more medical images 40.

In various embodiments, the one or more medical images 40 comprise single medical images, sets of medical images (e.g., medical images from different perspectives and/or captured via different types medical imaging technology), and/or a time series of medical images. In various embodiments, the one or more medical images are fluoroscopy images, ultrasound images, MRI images, and/or another type of medical images. In various embodiments, the one or more medical images 40 include a medical image of an oblique perspective.

At step 504, the computing entity 10 converts the one or more medical images 40 to the particular image format. For example, the computing entity 10 may comprise means, such as processor 102, memory 104, and/or the like, for converting the one or more medical images 40 to the particular image format. In various embodiments, the medical images 40 are obtained in an initial format. The computing entity 10 may determine and/or confirm whether the initial format of the medical images 40 is the particular image format. For example, the computing entity 10 (e.g., the image format converter 205) may compare a format name, file extension, metadata, the formatting of the image data, and/or the like of one or more of the medical images to a corresponding property of the particular image format. When the corresponding properties of the plurality of training images and the particular image format match, the computing entity 10 determines that the initial format is the particular image format, and no conversion is necessary. When the corresponding properties of the plurality of the training images and the particular image format do not match, the computing entity 10 determines that the initial format is not the particular image format and may cause conversion of the one or more medical images 40 to the particular image format. For example, when the initial format of the medical images is not the particular image format, the computing entity 10 may execute the image format converter 205 to cause the one or more medical images 40 to be converted to the particular image format. For example, the computing entity 10 comprises means, such as processor 102, memory 104, and/or the like, for converting the one or more medical images from an initial format to the particular image format.

In an example embodiment, the particular image format is a raster-graphics file format that supports lossless data compression, such as .png.

At step 506, the one or more medical images are provided as input to the object detection model 220 in the particular image format. For example, the computing entity 10 provides the one or more medical images 40, in the particular image format, to the object detection model 220 as input. For example, the computing entity 10 may cause the object detection model 220 to read in the one or more medical images 40 in the particular image format. In another example, the computing entity 10 may provide the one or more medical images 40 to an input layer of the object detection model 220, and/or the like. For example, the computing entity 10 comprises means, such as processor 102, memory 104, and/or the like for providing the one or more medical images 40 in the particular image format as input to the object detection model 220.

At step 508, the computing entity 10 causes the object detection model 220 to process the one or more medical images 40. For example, the computing entity 10 executes the object detection model 220 such that the object detection model 220 extracts processed image information from the one or more medical images 40. For example, the computing entity 10 executes the object detection model 220 such that the object detection model 220 converts the image data of the one or more medical images into processed image information. In various embodiments, the computing entity 10 comprises means, such as processor 102, memory 104, and/or the like, for executing the object detection model 220 to process the one or more medical images 40. In an example embodiment, execution of the object detection model 220 is performed as execution of a python script, for example, that includes the parameters and/or weights determined and/or learned through the training of the object detection model.

For example, the feature detector 222 may process the one or more medical images 40 to identify one or more object thereon and to classify the one or more objects. For example, the feature detector 222 determine that the one or more objects are present in the one or more medical images 40 and determine where in the one or more medical images 40 the objects are located. For example, the feature detector 222 may determine object localization information for one or more objects within the medical images 40. The feature detector 222 may further classify the one or more objects. For example, the feature detector 222 may determine object classification information for the one or more objects. In various embodiments, the feature detector 222 is configured to identify and classify multiple types or classes of objects in a medical image.

For example, the prediction model 224 may receive the object localization information and classification information and, possibly, the one or more medical images 40. The prediction model 224 may then generate prediction information based at least in part on the object localization information, classification information, and/or the one or more medical images 40. In various embodiments, the prediction information may provide a past and/or future trajectory of a medical device identified in the one or more medical images 40 (e.g., as indicated by the object localization information and classification information), whether the trajectory is a good trajectory (e.g., is expected to reach a goal location, is considered safe for the patient) or not a good trajectory (e.g., is not expected to reach the goal location, is not considered safe for the patient), and/or the like. In an example embodiment, the prediction information may provide a goal trajectory. The goal trajectory may be from a current position of a medical device identified in the one or more medical images 40 or from an initial position when medical device is not identified in the one or more medical images 40 (e.g., the medical device has not yet been inserted into the patient).

In an example embodiment, the prediction information may include an indication of a most advantageous level for medical intervention, a goal location for a trajectory of a medical device, and/or the like. For example, in an example embodiment, the feature detector 222 is trained to identify vertebral end plates in a medical image (e.g., an oblique fluoroscopy image). The polygons generated by the feature detector 222 that locate the vertebral end plates in the medical image (possibly along with corresponds labels/classifications) and the prediction model 224 may be trained to identify a most degenerated vertebra or vertebral end plate based on distances between vertebral end plates, a measure or degree of irregularity of the vertebral end plates, and/or the like. For example, the vertebral end plate having the greatest measure or degree of irregularity of the vertebral end plates in the medical image may be determined to be most advantageous level for medical intervention, a goal location for a trajectory of a medical device (e.g., an epidural needle), and/or the like.

Thus, in various embodiments, the processed image information includes localization information for one or more objects identified in the one or more medical images. The localization information indicates where in one or more images the respective objects are located and/or a relative location of the objects with respect to one another. In various embodiments, the processed image information further includes classification information for one or more objects identified in the one or more medical images. For example, the classification for each object identified in the one or more medical images 40 indicates a class determined and/or selected for the respective object (e.g., bony feature, vertebral pedicle, vertebral end plate, medical device, etc.). In various embodiments, the processed image information further includes prediction information.

In various embodiments, the object detection model 220 is executable on a central processing unit (CPU) of a smartphone, tablet, or laptop computer and provide the medical visualization in real time with respect to the capturing of the one or more medical images by the image capture device 30 and/or the obtaining of the one or medical images by the computing entity 10. For example, the object detection model 220 may be used to provide medical visualizations of a medical procedure while the medical procedure is being performed.

At step 510, the computing entity 10 receives the processed image information as output of the object detection model 220. For example, the computing entity 10 receives the processed image information as output of the object detection model 220. For example, the computing entity 10 may read and/or store the processed image information from an output of the object detection model 220. For example, the computing entity 10 comprises means, such as processor 102, memory 104, and/or the like for receiving the processed image information as output of the object detection model 220.

At step 512, the computing entity generates a graphical representation of the processed image information. In various embodiments, the graphical representation of the processed image information is an image layer (e.g., configured to be overlaid on at least one of the one or more medical images 40) that encodes and/or represents at least a portion of the processed image information. For example, FIG. 4C illustrates an example of a graphical representation 430 of the processed image information overlaid on a medical image 400. The graphical representation 430 of the processed image information is an image layer that includes pedicle locators 432A, 432B, 432C and a needle locator 434. Each of the pedicle locators indicates the location of a vertebral pedicle identified in the medical image 400. For example, the pedicle locators 432A, 432B, 432C represent the localization information of the identified bony features within the medical image 400. The needle locator 434 indicates the location of a tip of an epidural needle, which is an example of a medical device. For example, the needle locator 424 represents the localization information of the identified medical image within the medical image 400. In the illustrated embodiments, each of the pedicle locators 432A, 432B, 432C and the needle locator 434 are associated with a bounding rectangle and a confidence level.

For example, in various embodiments, the graphical representation engine 230 generates elements of an image layer that indicate the locations of each of the objects identified in the medical image, such as pedicle locators 432A, 432B, 432C and needle locator 434. The graphical representation engine 230 may then generate textual information corresponding to each of the locators, such as labels indicating respective classifications of the objects and/or other information such as the confidence level associated with the identification, location, and/or classification of a respective object. In various embodiments, the image layer may also include prediction information, such as a line or cone indicating a trajectory. In an example embodiment, the graphical representation engine 230 renders the image layer.

At step 514, the computing entity 10 causes display of the graphical representation of the processed image information. For example, the computing entity 10 causes display of the graphical representation of the processed image information overlaid on at least one of the one or more medical images 40. For example, the computing entity 10 may cause a display that is either part of the computing entity 10 (e.g., via user interface 108) or in communication with the computing entity 10 (e.g., via communication interface 106) to display the graphical representation of the processed image information. For example, the computing entity 10 may cause the display that is part of or in communication with the computing entity 10 to display a medical image of the one or more medical images and to display the graphical representation of the processed image information thereover (e.g., overlaid thereon). For example, a computing entity 10 comprises means, such as processor 102, memory 104, communication interface 106, user interface 108, and/or the like for causing a display to display the graphical representation of the processed image information, possibly as an image layer overlaid on a medical image of the one or more medical images 40.

For example, the display on which the graphical representation of the processed image information, possibly as an image layer overlaid on a medical image of the one or more medical images 40 may be in room in which a medical procedure is being performed. The medical image may be captured prior to and/or during performance of the medical procedure. A healthcare provider performing the medical procedure may use the displayed graphical representation of the processed image information as a guide or feedback during performance of the medical procedure. For example, the healthcare provider may determine whether to continue along a particular trajectory or change the trajectory of a medical device based on the displayed graphical representation of the processed image information. For example, the graphical representation of the processed image information may be displayed in real-time with respect to the capture of the medical image(s) and/or the obtaining of the medical images by the computing entity 10 such that the healthcare provider can use the displayed graphical representation of the processed image information for real-time feedback regarding performance of the medical procedure.

For example, the display of the graphical representation of the processed image information may be updated (in real-time) when one or more new medical images are captured (e.g., by the image capture device 30) and/or obtained by the computing entity 10. In an example embodiment, an updated display of the graphical representation of the processed image information may include an indication of an actual trajectory of a medical device present in the medical images. For example, when a medical device is identified in a first medical image captured at a first time, and then in a second medical image captured at a second time (which is later than the first time), when the display of the graphical representation of the processed image information is updated based on second medical image, the displayed graphical representation may include an indication of the previous position(s) of the medical device (e.g., epidural needle tip) and the current position of the medical device so that the healthcare provider can see the change in position of the medical device since the previous image capture.

### IV. Example Computing Entity

The computing entity 10 of an example embodiment may be embodied by or associated with a variety of computing devices including, for example server, server bank, part of a cloud-based computing environment, desktop computer, laptop, tablet, smartphone, and/or the like. In this regard, FIG. 6 depicts an example computing entity 10 that may be embodied by various computing devices including those identified above. As shown, the computing entity 10 of an example embodiment may include, may be associated with, or may otherwise be in communication with a processor 102 and a memory device 104 and optionally a communication interface 106, and/or a user interface 108.

In some embodiments, the processor 102 (and/or co-processors or any other processing circuitry assisting or otherwise associated with the processor) may be in communication with the memory device 104 via a bus for passing information among components of the computing entity. The memory device 104 may be non-transitory and may include, for example, one or more volatile and/or non-volatile memories. In other words, for example, the memory device may be an electronic storage device (e.g., a non-transitory computer readable storage medium) comprising gates configured to store data (e.g., bits) that may be retrievable by a machine (e.g., a computing device like the processor 102). The memory device may be configured to store information, data, content, applications, instructions, or the like for enabling the computing entity 10 to carry out various functions in accordance with an example embodiment of the present invention. For example, the memory device could be configured to buffer input data for processing by the processor. Additionally or alternatively, the memory device could be configured to store instructions for execution by the processor. For example, the memory device may store program code and/or executable instructions configured to, when executed by the processor, cause the processor to receive and analyze and/or process medical images (e.g., using the trained object detection model) so as to determine and/or extract information therefrom. In another example, the memory device may store program code and/or executable instructions configured to, when executed by the processor, cause the processor to train the object detection model based on a plurality of segmented and labeled training images.

As described above, the computing entity 10 may be embodied by a computing device. However, in some embodiments, a respective computing entity may be embodied as a chip or chip set. In other words, the computing entity may comprise one or more physical packages (e.g., chips) including materials, components and/or wires on a structural assembly (e.g., a baseboard). The structural assembly may provide physical strength, conservation of size, and/or limitation of electrical interaction for component circuitry included thereon. The computing entity may therefore, in some cases, be configured to implement an embodiment of the present invention on a single chip or as a single “system on a chip.” As such, in some cases, a chip or chipset may constitute means for performing one or more operations for providing the functionalities described herein.

The processor 102 may be embodied in a number of different ways. For example, the processor 102 may be embodied as one or more of various hardware processing means such as a coprocessor, a microprocessor, a controller, central processing unit (CPU), graphics processing unit (GPU), a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. As such, in some embodiments, the processor 102 may include one or more processing cores configured to perform independently. A multi-core processor may enable multiprocessing within a single physical package. Additionally or alternatively, the processor 102 may include one or more processors configured in tandem via the bus to enable independent execution of instructions, pipelining and/or multithreading.

In an example embodiment, the processor 102 may be configured to execute instructions stored in the memory device 104 or otherwise accessible to the processor. Alternatively or additionally, the processor may be configured to execute hard coded functionality. As such, whether configured by hardware or software methods, or by a combination thereof, the processor may represent an entity (e.g., physically embodied in circuitry) capable of performing operations according to an embodiment of the present invention while configured accordingly. Thus, for example, when the processor is embodied as an ASIC, FPGA or the like, the processor may be specifically configured hardware for conducting the operations described herein. Alternatively, as another example, when the processor is embodied as an executor of software instructions, the instructions may specifically configure the processor to perform the algorithms and/or operations described herein when the instructions are executed. However, in some cases, the processor may be a processor of a specific device (e.g., a pass-through display or a mobile terminal) configured to employ an embodiment of the present invention by further configuration of the processor by instructions for performing the algorithms and/or operations described herein. The processor may include, among other things, a clock, an arithmetic logic unit (ALU) and logic gates configured to support operation of the processor.

In some embodiments, the computing entity 10 may include a user interface 108 that may, in turn, be in communication with the processor 102 to provide output to the user, such as at least a portion of the processed image information and/or a graphical representation thereof. As such, the user interface may include a display and, in some embodiments, may also include a keyboard, a mouse, a joystick, a touch screen, touch areas, soft keys, a microphone, a speaker, or other input/output mechanisms. Alternatively or additionally, the processor may comprise user interface circuitry configured to control at least some functions of one or more user interface elements such as a display and, in some embodiments, a speaker, ringer, microphone and/or the like. The processor and/or user interface circuitry comprising the processor may be configured to control one or more functions of one or more user interface elements through computer program instructions (e.g., software and/or firmware) stored on a memory accessible to the processor 102 (e.g., memory device 104, and/or the like).

The computing entity 10 may optionally include a communication interface 106. The communication interface may be any means such as a device or circuitry embodied in either hardware or a combination of hardware and software that is configured to receive and/or transmit data from/to a network and/or any other device or module in communication with the computing entity 10. In this regard, the communication interface may include, for example, an antenna (or multiple antennas) and supporting hardware and/or software for enabling communications with a wireless communication network. Additionally or alternatively, the communication interface may include the circuitry for interacting with the antenna(s) to cause transmission of signals via the antenna(s) or to handle receipt of signals received via the antenna(s). In some environments, the communication interface may alternatively or also support wired communication. As such, for example, the communication interface may include a communication modem and/or other hardware/software for supporting communication via cable, digital subscriber line (DSL), universal serial bus (USB) or other mechanisms. For example, the computing entity may be configured to obtain training images and/or medical images via a communication interface 106.

As described above, FIGS. 3 and 5 illustrate flowcharts of a present embodiment of FIG. 1, computing entity 10, methods, and computer program products according to an example embodiment. It will be understood that each block of the flowcharts, and combinations of blocks in the flowcharts, may be implemented by various means, such as hardware, firmware, processor, circuitry, and/or other devices associated with execution of software including one or more computer program instructions. For example, one or more of the procedures described above may be embodied by computer program instructions. In this regard, the computer program instructions which embody the procedures described above may be stored by the memory device 404 of computing entity 10 employing an embodiment of the present invention and executed by the processor 102 of the computing entity 10. As will be appreciated, any such computer program instructions may be loaded onto a computer or other programmable apparatus (e.g., hardware) to produce a machine, such that the resulting computer or other programmable apparatus implements the functions specified in the flowchart blocks. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart blocks. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart blocks.

Accordingly, blocks of the flowcharts support combinations of means for performing the specified functions and combinations of operations for performing the specified functions for performing the specified functions. It will also be understood that one or more blocks of the flowcharts, and combinations of blocks in the flowcharts, can be implemented by special purpose hardware-based computer systems which perform the specified functions, or combinations of special purpose hardware and computer instructions.

In some embodiments, certain ones of the operations above may be modified or further amplified. Furthermore, in some embodiments, additional optional operations may be included. Modifications, additions, or amplifications to the operations above may be performed in any order and in any combination.

### V. Technical Advantages

Conventional lumbar transforaminal epidural steroid injections, guided by fluoroscopy, are a mainstay in managing lumbar pain among interventional anesthesiologists in the United States. They are lauded for their effectiveness in short-term pain relief. However, patients with lumbar spine degeneration and stenosis often present complexities that demand greater precision in the procedure. The potential for complications, although generally minor, such as vasovagal reactions, pain exacerbation, and injection site soreness, underscores a critical need for enhanced accuracy in needle placement. This accuracy is not just a matter of improving the procedure's effectiveness but is also a consideration for patient safety. Moreover, a level where a patient is experiencing and/or reporting systems may not be the most advantageous level for medical intervention. However, conventional techniques for determining a medical intervention level are based on patient symptom reporting. Therefore, technical challenges exist regarding performance of epidural injections and other similar medical procedures.

Various embodiments provide technical solutions to these technical challenges. For example, various embodiments provide for using an object detection model to identify a goal location and/or a most advantageous level for medical intervention of a patient's spine. This enables a selection of the level for medical intervention (e.g., which level of the patient's spine) to be treated via the epidural injection to provide improved pain relief to the patient. Moreover, various embodiments provide pre-medical procedure and/or real-time during the medical procedure feedback to the healthcare provider and/or team performing the medical procedure. In some embodiments, the real-time feedback provided by various embodiments (e.g., the medical visualization provided by displaying a graphical representation of at least a portion of the processed image information overlaid, for example, on the medical image) enables healthcare providers to adjust a trajectory of a medical device, such as an epidural needle, in real-time to provide a higher probability of the medical device reaching the goal location in a manner that is considered safe for the patient. These improvements are provided, at least in part, through the use of the particular image format, the training of the feature detector to detect multiple classes of objects in a single image. These features of various embodiments enable the object detection model to be able to extract and/or generate processed image information from the one or more medical images quickly, such that real-time feedback may be provided to the healthcare provider/team, and with minimal computational resources (e.g., the object detection model may be executed by a CPU, smartphone processor, tablet processor, laptop processor, and/or the like). Additionally, the use of the prediction model to generate and provide predictions regarding various types of trajectories, whether a trajectory is a good trajectory or not, a most advantageous level for medical intervention, a goal location of a medical device for performance of a medical procedure, and/or the like provides technically improved tools for medical providers. Therefore, various embodiments provide technical advantages over conventional techniques for identifying a goal location for a medical intervention and guidance of a medical device to the goal location.

### VI. Conclusion

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for providing a medical visualization, the method comprising:
providing, by one or more processors of a computing entity, a medical image in a particular image format to an input of a trained object detection model;
causing, by the one or more processors, the trained object detection model to be executed to process the medical image to generate processed image information, the processed image information configured to at least identify one or more bony features within the medical image;
receiving, by the one or more processors, the processed image information from an output of the trained object detection model; and
causing, by the one or more processors, display of the medical image with a representation of at least a portion of the processed image information overlaid on the medical image,
wherein the trained object detection model was trained using a training process comprising:
performing an instance segmentation on a plurality of training images, each of the plurality of training images being in the particular image format;
assigning semantic labels to each object identified via the instance segmentation; and
using at least a portion of the plurality of training images, the instance segmentation of the at least a portion of the plurality of training images, and the semantic labels assigned to objects identified in the at least a portion of the plurality of training images to train an object detection model.

2. The method of claim 1, wherein the processed image information is further configured to identify at least one medical device within the medical image.

3. The method of claim 2, wherein the at least one medical device is an epidural needle.

4. The method of claim 2, wherein the medical image is one of a time series of medical images and the processed image information includes trajectory information indicating a trajectory of the at least one medical device, the trajectory being at least one of a past trajectory of the at least one medical device, a future trajectory of the at least one medical device, or a goal trajectory of the at least one medical device, and causing display of the representation of the at least a portion of the processed image information overlaid on the medical image comprises causing display of a representation of the trajectory on the medical image.

5. The method of claim 4, wherein the trajectory information further indicates whether the trajectory of the at least one medical device is likely to result in the at least one medical device reaching a goal destination.

6. The method of claim 1, wherein the plurality of training images comprises one or more time series of training images that are associated with respective trajectory labels.

7. The method of claim 1, wherein the processed image information is further configured to identify a bony feature of the one or more bony features which exhibits the most irregularity of the one or more bony features.

8. The method of claim 1, wherein the trained object detection model is a single step object detection model.

9. The method of claim 1, wherein the display of the medical image with the representation of the at least a portion of the processed image information overlaid on the medical image occurs in real-time with respect to the providing of the medical image to the trained object detection model.

10. The method of claim 1, wherein the particular image format is .png.

11. The method of claim 1, wherein the particular image format is a raster-graphics file format that supports lossless data compression.

12. The method of claim 1, wherein the trained object detection model is executed via a CPU of the computing entity.

13. The method of claim 1, wherein the object detection model comprises a feature detector and a prediction model.

14. The method of claim 13, wherein the feature detector is trained to identify and classify objects within the medical image and the prediction model is configured to generate a prediction based at least in part on identification and classification of objects in the medical image performed by the feature detector.

15. The method of claim 14, wherein the prediction comprises at least one of a goal location for a medical device for a medical procedure or a most advantageous level for medical intervention.

16. A computing entity comprising at least one processor and a memory, the memory storing executable instructions configured to, when executed by the at least one processor, cause the computing entity to perform at least:
providing a medical image in a particular image format to an input of a trained object detection model;
causing the trained object detection model to be executed to process the medical image to generate processed image information, the processed image information configured to at least identify one or more bony features within the medical image;
receiving the processed image information from an output of the trained object detection model; and
causing display of the medical image with a representation of at least a portion of the processed image information overlaid on the medical image,
wherein the trained object detection model was trained using a training process comprising:
performing an instance segmentation on a plurality of training images, each of the plurality of training images being in the particular image format;
assigning semantic labels to each object identified via the instance segmentation; and
using at least a portion of the plurality of training images, the instance segmentation of the at least a portion of the plurality of training images, and the semantic labels assigned to objects identified in the at least a portion of the plurality of training images to train an object detection model.

17. The computing entity of claim 16, wherein the processed image information is further configured to identify at least one medical device within the medical image.

18. The computing entity of claim 17, wherein the medical image is one of a time series of medical images and the processed image information includes trajectory information indicating a trajectory of the at least one medical device, the trajectory being at least one of a past trajectory of the at least one medical device, a future trajectory of the at least one medical device, or a goal trajectory of the at least one medical device, and causing display of the representation of the at least a portion of the processed image information overlaid on the medical image comprises causing display of a representation of the trajectory on the medical image.

19. The computing entity of claim 16, wherein the processed image information is further configured to identify a bony feature of the one or more bony features which exhibits the most irregularity of the one or more bony features.

20. A computer program product comprising at least one non-transitory computer-readable medium, the computer-readable medium storing computer-executable instructions configured to, when executed by a processor of a computing entity, cause the computing entity to perform
providing a medical image in a particular image format to an input of a trained object detection model;
causing the trained object detection model to be executed to process the medical image to generate processed image information, the processed image information configured to at least identify one or more bony features within the medical image;
receiving the processed image information from an output of the trained object detection model; and
causing display of the medical image with a representation of at least a portion of the processed image information overlaid on the medical image,
wherein the trained object detection model was trained using a training process comprising:
performing an instance segmentation on a plurality of training images, each of the plurality of training images being in the particular image format;
assigning semantic labels to each object identified via the instance segmentation; and
using at least a portion of the plurality of training images, the instance segmentation of the at least a portion of the plurality of training images, and the semantic labels assigned to objects identified in the at least a portion of the plurality of training images to train an object detection model.
